(19) United States
(12) Patent Application Publication
(10) Pub. No.: US 2005/0192588 A1
(43) Pub. Date: Sep. 1, 2005

(54) Instrumentation and method for prosthetic knee

(76) Inventor: Daniel X. Garcia, Battle Creek, MI (US)

Correspondence Address: WARNER NORCROSS & JUDD LLP, 900 FIFTH THIRD CENTER, 111 LYON STREET, N.W., GRAND RAPIDS, MI, 49503-2487, US

(21) Appl. No.: 11/068,115

(22) Filed: Feb. 28, 2005

Related U.S. Application Data
(60) Provisional application No. 60/548,651, filed on Feb. 27, 2004.

(51) Int. Cl. A61B 17/58

(52) U.S. Cl. 606/88

(57) ABSTRACT

Instrumentation for guiding a surgeon in performing a unicompartmental knee replacement includes a tibial block having a guide, and a milling tool adapted to engage the guide, such that the surgeon can mill the desired tibial bone bed by directing the milling tool along the guide. Further including a femoral jig having a cutting slot, a guide, and a milling tool adapted to engage the guide, such that a surgeon can cut a portion of the femur through the slot, and mill the femoral bone bed by directing the milling tool about the guide. A femoral trial removal clamp facilitates in removing the femoral trial prosthesis, and a spreader compression clamp aids in compressing the final prostheses as the bone cement cures.

This application claims priority from U.S. Provisional Application Ser. No. 60/548,651, filed on Feb. 27, 2004.

### BACKGROUND OF THE INVENTION

The present invention is related to a prosthetic device, and more particularly to instrumentation to aid a surgeon in the insertion of a prosthetic knee and the associated method.

Knee replacement surgery typically involves removal of all or a portion of the existing knee joint, and the insertion of artificial prostheses (or components). Total knee replacement (TKR) is a surgical procedure that has been successfully performed on humans for over three decades. The orthopedic surgeon utilizes a series of sequential instruments and guides to cut and shape the patient's host bone on which implants are fixed, resulting in replacement of the worn, arthritic surfaces of all three compartments of the knee with smooth articulating artificial surfaces. However, because a significant segment of knee replacement candidates have worn out or damaged only one of the three knee compartments, TKR is often unnecessarily invasive and therefore undesirable.

Newer innovation has addressed this problem with the implementation of unicompartmental or unicondylar knee replacement (UKR). This procedure is much less invasive, because only the single worn knee compartment is replaced. One leading implant currently used in this less invasive procedure is the Repicci Unicompartmental Knee Replacement, manufactured by Biomet, Inc. The Repicci replacement is particularly non-invasive, in that the prostheses are manufactured to be inlayed into bone beds milled into portions of the host bones, as opposed to other methods that require resecting an entire layer of bone from a knee compartment to “onlay” the prosthetics over the surfaces of the knee compartment.

Unfortunately, UKR and particularly the Repicci UKR are currently underutilized by surgeons due to the difficulty in preparing the host bones for the procedure. A few basic instruments exist to aid in positioning the knee implants, such as the Vanguard tibial extramedullary alignment jig. However, the surgery continues to be difficult for surgeons to perform, particularly because the procedure must be performed almost entirely by freehand milling and drilling of the host bones. This results in a very tedious and time consuming operation, a steep learning curve for doctors, and optimal candidates receiving an overly invasive operation, or no operation at all.

### SUMMARY OF THE INVENTION

The aforementioned problems are overcome by the present invention wherein a series of sequential instruments are used to guide bone cutting and milling tools for implanting UKR components. In one embodiment, the instruments are designed specifically to aid in the implantation of the inlayed Repicci UKR components.

In one embodiment, the present invention includes a tibial block for use in conjunction with a standard tibial extramedullary alignment jig. The jig is used to align the tibial block with the host bone, so that the block can be pinned to the knee in a conventional manner. One surface of the block includes a guide or a set of guides for engaging a first portion of a milling tool, such that when the first portion engages the guide, the cutting portion or burr of the milling tool is in direct contact with the host bone. In a further embodiment, the milling tool may be specifically adapted to engage the guide by including a burr that is perpendicular to the handle of the tool and one or more adjustable pegs. As the surgeon directs the first portion of the tool, including the pegs, along the guides, the perpendicular burr mills out the exact pattern in the host bone.

In another embodiment, the present invention further includes a femoral jig. In this embodiment, the femoral jig is preferably pinned to the femur, using the tibial block to guide its alignment. The femoral jig preferably includes a cutting slot portion that may be attached to the femoral jig as a separate piece. The cutting slot is capable of receiving a standard surgical saw blade or a punch, to guide the resection of a lower portion of the host femur. The femoral jig also includes a perimeter guide, or groove, for engaging an adjustable first portion of a power milling tool, such that when the first portion of the tool is directed along the groove, the burr of the milling tool mills out a portion of the host femur in the same pattern. In one embodiment, a sleeve attaches to the milling tool for engaging the perimeter groove. The sleeve adjustably attaches about the first portion of the milling tool and preferably includes a number of arms for engaging the perimeter guide, or groove.

The present invention may also include an insert for use with the femoral milling jig. The insert may be a plate that is positioned inside the perimeter guide of the femoral jig, and includes a hole that receives a conventional drill, and a slot or slots that receive a conventional saw blade or a punch. The hole may further include a tubular protrusion having a desired depth to act as a drill guide and stop.

In another embodiment, the present invention includes a femoral trial component removal clamp for removing the femoral trial component after it has been impacted into the prepared bone bed. The removal clamp includes opposing sharp, angled jaw members, which can be forced under opposite edges of the femoral trial component to remove the femoral trial component without damaging the thin perimeter host bone.

In yet another embodiment, a spreader compression clamp is used to apply pressure to the finial implants after they have been inserted in the host bone. The clamp includes a first prong for contacting the tibial implant and a second prong for contacting the femoral implant. The clamp is used to apply pressure to the implants simultaneously and evenly as the bone cement cures. The clamp prevents the implants from seating in an improper position and from shifting as the cement cures.

The present invention also includes a method for performing a unicompartmental knee replacement, including the steps of: a) pinning a tibial block including a guide or guides to the host tibia; b) adapting a milling tool to engage the guide on the tibial block; c) pinning a femoral milling jig to the host femur; d) preparing the host femur to receive the femoral implant using the femoral milling jig to guide cutting and milling tools; e) milling the surface of the host tibia by directing the milling tool along the tibial block guide; f) inserting trial femoral and tibial implants into the prepared host femur and tibia and removing the femoral trial with a femoral trial removal clamp; and g) compressing the final tibial and femoral implants with a spreader compression clamp.

The present invention provides a set of instrumentation to aid surgeons in implanting UKR components and provides numerous other advantages, such as: reducing the time required for cutting and milling the host bones; decreasing learning time for new surgeons; increasing the accuracy and reproducibility of the milling of the host bones, improving the positioning of the implants—a crucial factor in the longevity of the UKR leading to more successful surgeries; and increasing the availability of the UKR procedure by making the implantation more “surgeon friendly.”

These and other objects, advantages, and features of the invention will be more fully understood and appreciated by reference to the detailed description of the current embodiment and the drawings.

### BRIEF DESCRIPTION OF THE DRAWINGS

FIG. 1 is a side view of a tibial extramedullary alignment jig including the tibial block attached to a tibia.

FIG. 2 is a front view of a knee joint including a femoral component and a tibial component following UKR.

FIG. 3 is a side view of the tibial block attached to the knee.

FIG. 4 is a perspective view of one embodiment of the tibial block.

FIG. 5 is a side view of the tibial block attached to the knee and a burr engaged with the tibial block.

FIG. 6 is an exploded view of a tibial block and a burr.

FIG. 7 is a perspective view of a burr according to the present invention engaged with the tibial block and the tibia.

FIG. 8 is a top view of the tibial block and burr according to one embodiment of the present invention.

FIG. 9 is a perspective view of the tibial block and burr according to one embodiment of the present invention.

FIG. 10 is an exploded view of the tibia and the tibial component.

FIG. 11 is a side view of the spreader compression clamp engaged with the tibia and femur.

FIG. 12 is a perspective side view of the femoral jig attached to the femur showing various positions of the milling tool.

FIG. 13 an exploded view of the femoral jig and a milling tool.

FIG. 14 is a perspective view of the femoral jig attached to the femur and a saw for resection of the distal femur.

FIG. 15 is a perspective view of a partially prepared femur.

FIG. 16 is a perspective view of the femoral jig attached to the femur and a milling tool with a first prong engaged with the femoral jig.

FIG. 17 is a perspective view of the femoral jig attached to the femur and a milling tool with a second prong engaged with the femoral jig.

FIG. 18 is a front view of the femoral jig attached to the femur showing the path of the milling tool with a first prong engaged with the femoral jig.

FIG. 19 is a front view of the femoral jig attached to the femur showing the path of the milling tool with a second prong engaged with the femoral jig.

FIG. 20 is an exploded view of the partially prepared femur and the femoral jig insert.

FIG. 21 is a perspective view of the femoral jig insert and a punch.

FIG. 22 is a perspective view of a prepared femur.

FIG. 23 is an exploded view of the prepared femur and the femoral component.

FIG. 24 is a front view of the installed UKR components.

FIG. 25 is a side view of the installed UKR components.

FIG. 26 is a side view of the femoral trial removal clamp.

FIG. 27 is a front view of the femoral trial removal clamp.

FIG. 28 is a side view of an alternative embodiment of the spreader compression clamp.

FIG. 29 is a side view of another alternative embodiment of the spreader compression clamp.

### DETAILED DESCRIPTION OF THE CURRENT EMBODIMENT

The present invention is directed to instruments used in aiding a surgeon in a unicompartmental knee replacement. As shown in the drawings, the present invention may include a tibial block 20, and a milling tool 22 adapted to engage the tibial block 20. The present invention may also include a femoral jig 30, a femoral jig insert 32, a milling tool 34 adapted to engage the femoral jig 30, a femoral trial removal clamp 36, and a spreader compression clamp 38. In operation, the tibial block 20 and adapted milling tool 22 are used to guide a surgeon in preparing a host tibia bone bed 44 for insertion of a tibial prosthesis 12. The femoral jig 30 and adapted milling tool 34 are used to guide a surgeon in preparing a host femur bone bed 60 for the insertion of a femoral prosthesis 16.

The present invention is described herein for use in the preparation of host bones for a unicompartmental knee replacement, specifically for use with the Repicci unicompartmental knee replacement. It should be noted, however, that all or a portion of the present invention could be adapted for use with other unicompartmental knee replacements.

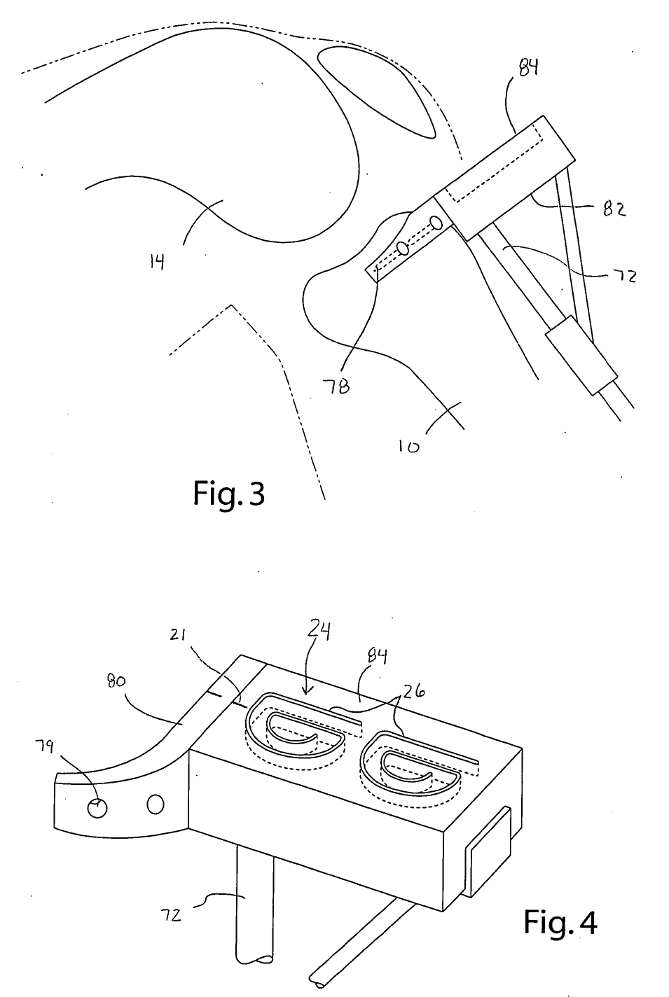

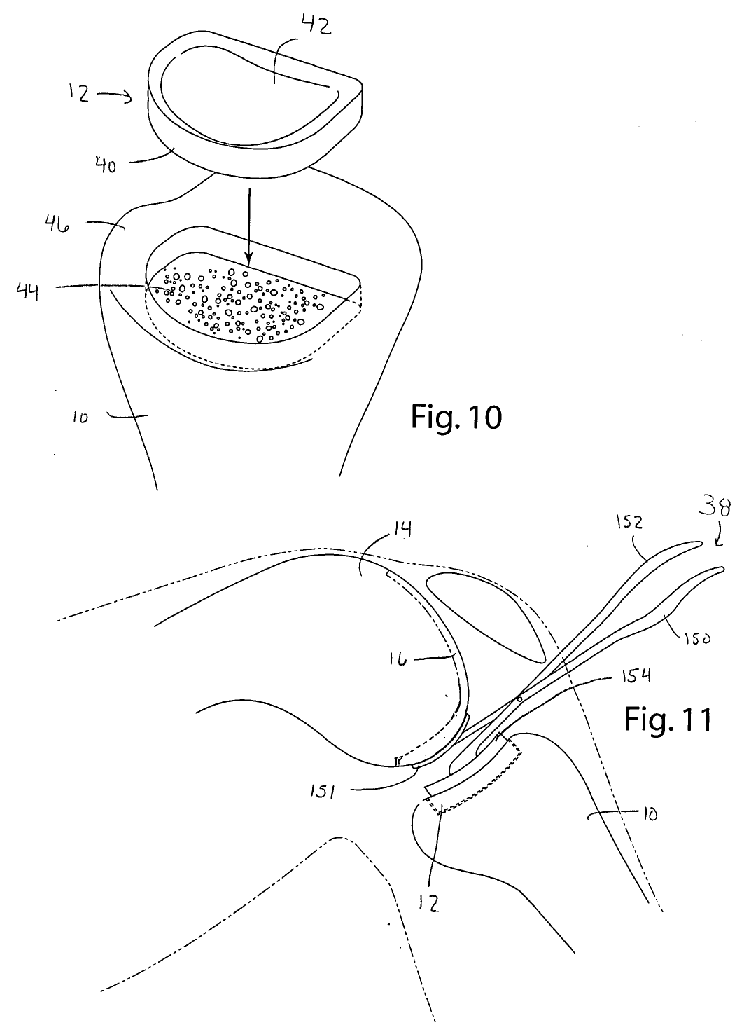

As shown in FIG. 2, the Repicci UKR includes two prosthetic components, a tibial component 12 and a femoral component 16. The tibial component 12 is generally a high molecular weight polyethylene pad. As more particularly shown in FIG. 10, the tibial component 12 has a generally semi-circular shape, a flat lower surface 40, and a slightly concave upper surface 42. It is designed to be placed in a trough or bone bed 44 that is milled out of the upper surface 46 of the tibia 10. As more particularly shown in FIG. 23, the femoral component 16 is L-shaped, but with a rounded corner 48 on the outer surface 50 and the inner surface 52. A protrusion or central peg 54 extends from the inner surface 52, along with a pair of fins 56 that extend along opposing sides of the protrusion 54. The femoral implant 16 is designed to be fit with the host femur 14, after the femur 14 has been milled to accommodate for the shape of the prosthesis and to receive the protrusion 54 and fins 56. In general, in order to prepare the femur 14, a portion of the lower femur surface 58 is resected, and a trough or bed 60 is milled out of the front surface 62 of the femur 14. Finally, a slot 64 is cut or punched into the front surface 62 to receive the protrusion 54 and fins 56.

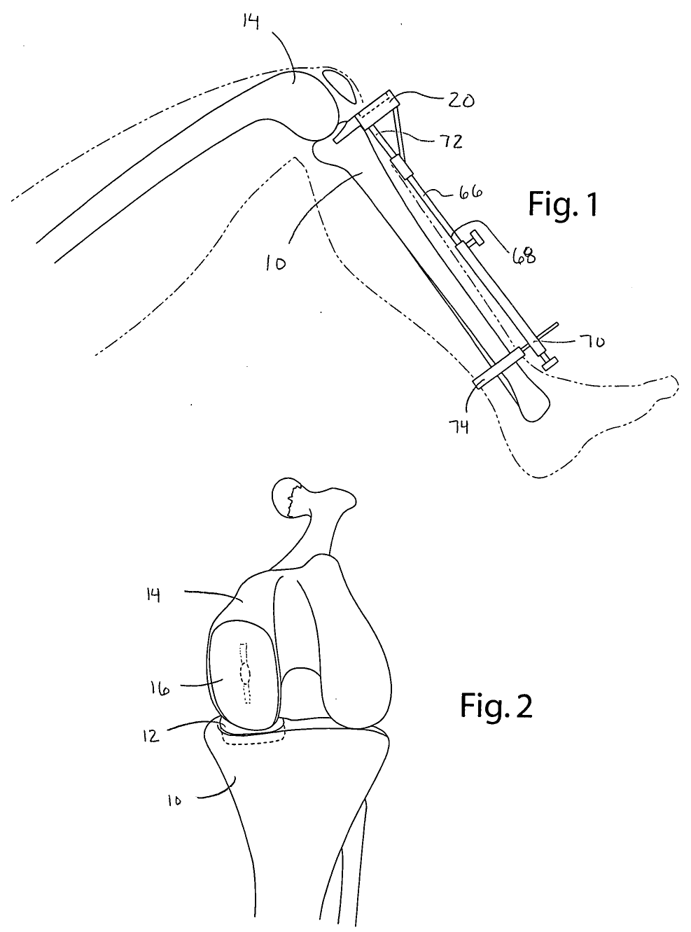

As shown in FIGS. 1, 3, and 4, the tibial block 20, is generally a rectangular block that is attached to the top of a conventional tibial extramedullary alignment jig 66, such as the Vanguard jig made by Biomet, Inc. The jig 66 is conventional, and therefore will not be described in great detail. Suffice it to say that the jig 66 includes a telescoping shaft 68 having a first end 70 and a second end 72. The first end 70 is affixed to the ankle region of the patient's leg, for instance, by a cuff 74 that may be tightened about the patient's ankle. The second end 72 is conventionally attached to a lower surface 82 of the rectangular tibial block 20. The block 20 includes a front portion 80 that is contoured to fit against the exterior surface of the upper tibia 10. This front portion 80 attaches directly to the upper tibia 10, for instance, by a plurality of pins 78 that extend through holes 79 in the front portion 80 to secure the block to the tibia 10 in standard fashion. A standard stylus (not shown) is optionally attachable to the block 20 to guide the pinning of the block at the appropriate height. As shown in FIG. 4, the tibial block may include a score line 21 to mark the midline of the tibial component bed, assisting in marking the weight bearing midline of the component, depending on the size of the tibial component chosen.

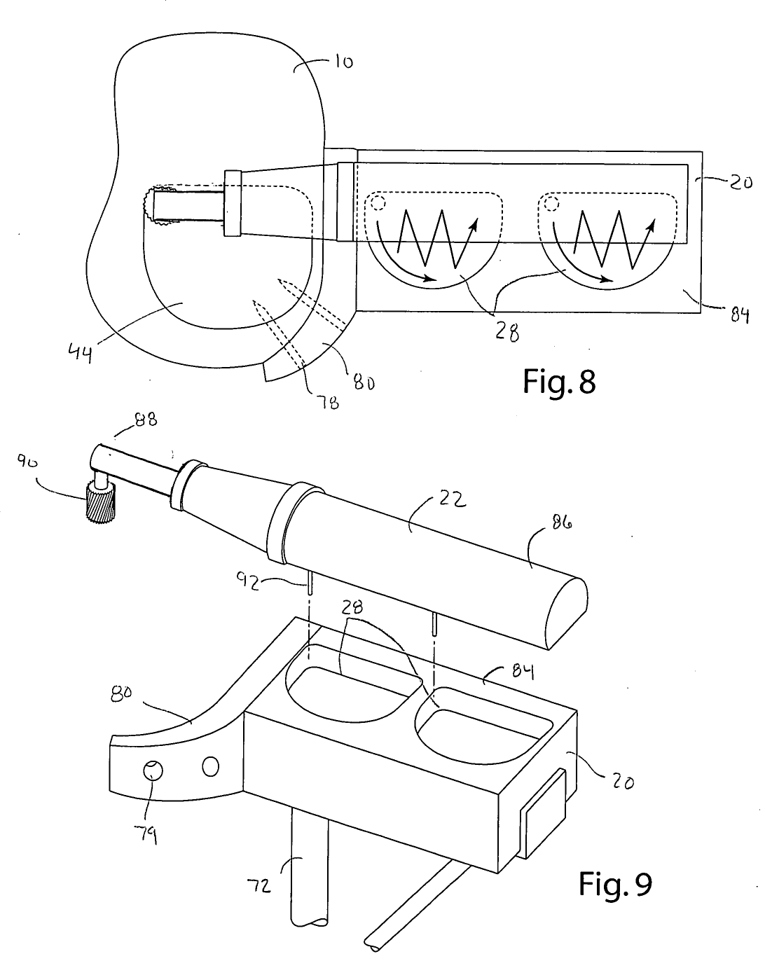

The tibial block 20 further includes a guide 24 on at least one surface, preferably the upper surface 84. FIG. 4-7 show a first embodiment of the guide, wherein the guide includes a pair of grooves 26 in the upper surface 84, shaped in a spiral-like pattern. As discussed below, the grooves 26 follow a path that is shaped similar to the desired tibial bone bed 44 to be milled. FIGS. 8 and 9 show a second embodiment of the guide 24, wherein the guide 24 includes two semi-circular shaped troughs 28. The troughs 28 are approximately the same size and shape as the tibial bone bed 44 to be milled. In one embodiment, the present invention may include a number of tibial blocks to choose from including either of the above mentioned guides or an alternative guide pattern as desired. The various blocks 20 may be interchangeable on the jig 66, with each having guides of different sizes to correspond with the various sizes of the Repicci tibial implants 12.

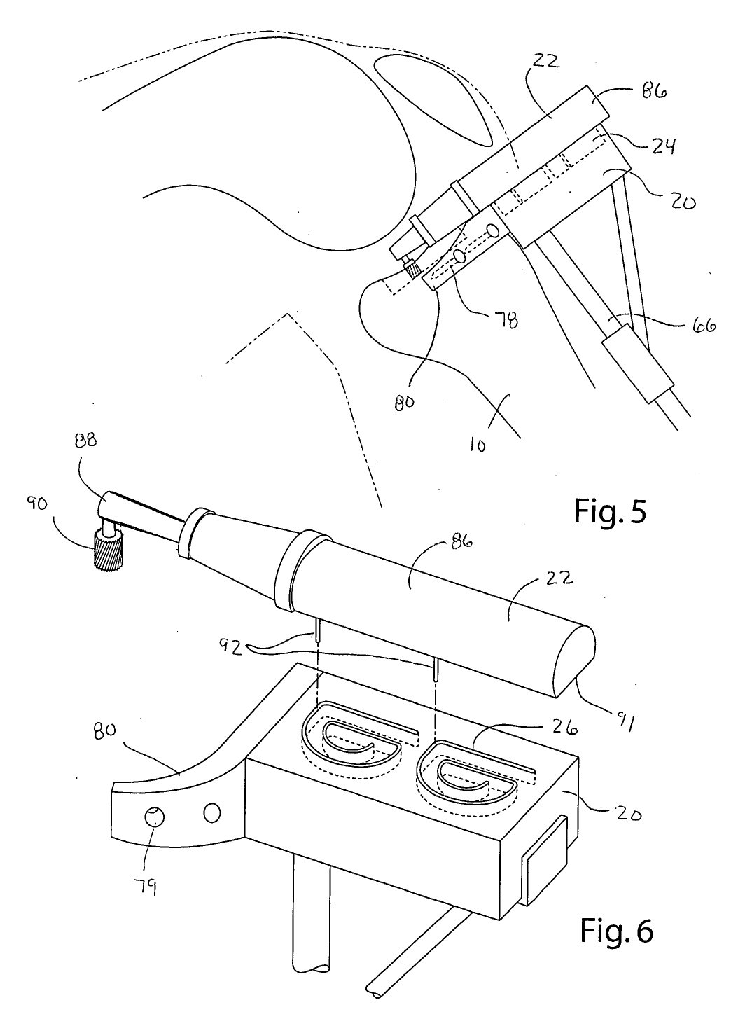

As shown in FIGS. 5-9, a power milling tool 22 is adapted to engage the guides 24 on the tibial block 20. The milling tool 22 may include a powered handle 86, a neck 88, and a burr 90 that extends from the neck 88 and is angled at 90 degrees with the powered handle 86. The interior of the handle (not shown) includes a conventional motor for actuating a rotary drive shaft. The drive shaft may include a gear assembly to connect the angled burr 90 to the drive shaft for providing the burr 90 with rotation. As illustrated, the lower surface 91 of the handle 86 is flat, such that it may sit flush on top of the tibial block 20 when engaged with the tibial block 20. The remaining surfaces of the handle 86 and neck 88 may have a variety of shapes and sizes. The handle 86 further includes a pair of guide pegs 92. As shown, the pegs 92 are integrally formed or assembled with the powered handle 86 by extending into mounting holes on the handle 86. Alternatively, the pegs 92 may be part of a separate assembly that fits or snaps on to the power handle 86, such as a clamping ring that is tightened about the handle 86. The pegs 92 are short, parallel protrusions that may have an adjustable length by adjusting the depth the pegs 92 extend into the mounting holes. Alternatively, the adjustable length may be provided by separate attachments that fit on the power handle, or by providing multiple pegs 92 of different lengths. The pegs 92 may also be adjustable longitudinally along the power handle 86, for instance, by attaching the pegs 92 as a separate assembly, such as the above-mentioned ring, that can slide along the handle 86. The guide pegs 92 are matched and sized to fit within the guide 24 on the tibial block 20, such that the pegs 92 can be directed by a surgeon along the path of the grooves 26 of the first tibial block embodiment, or within the troughs 28 of the second embodiment.

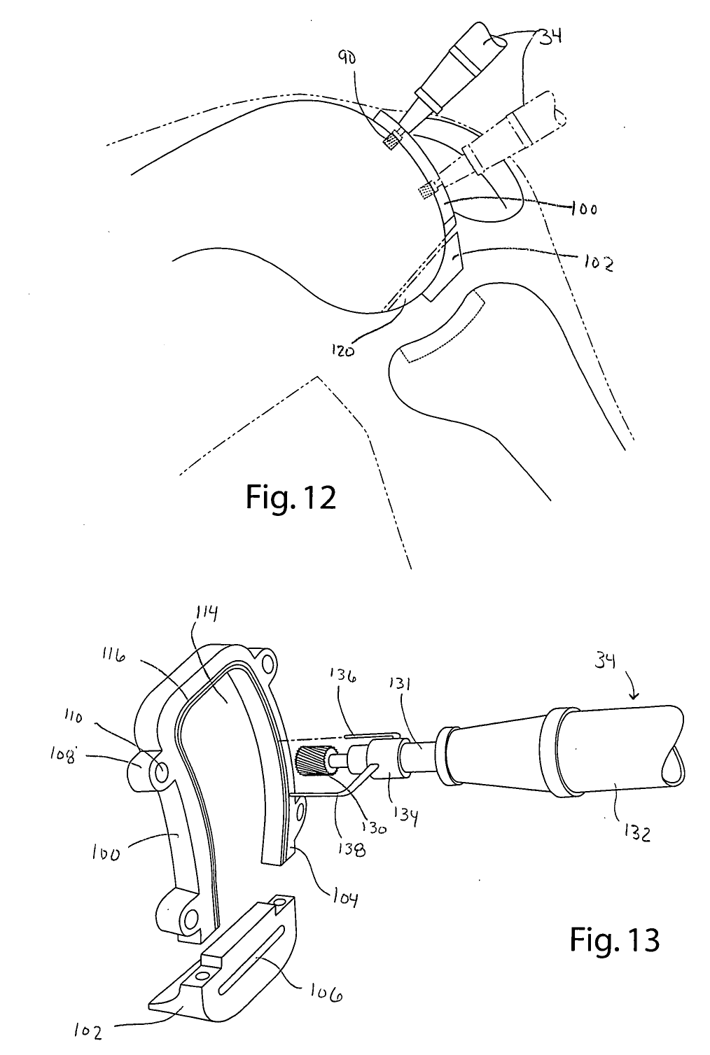

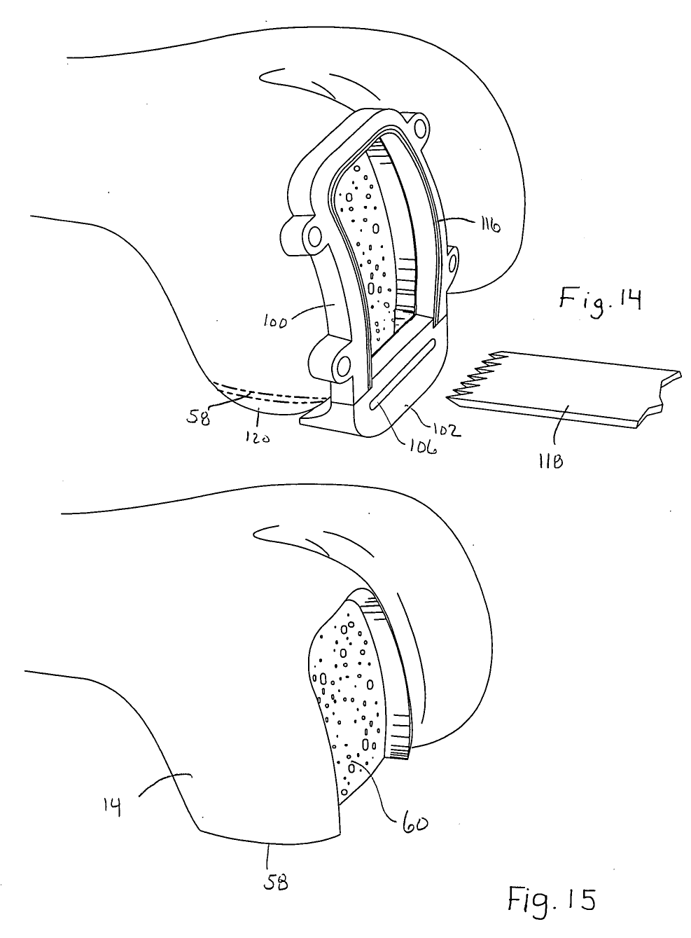

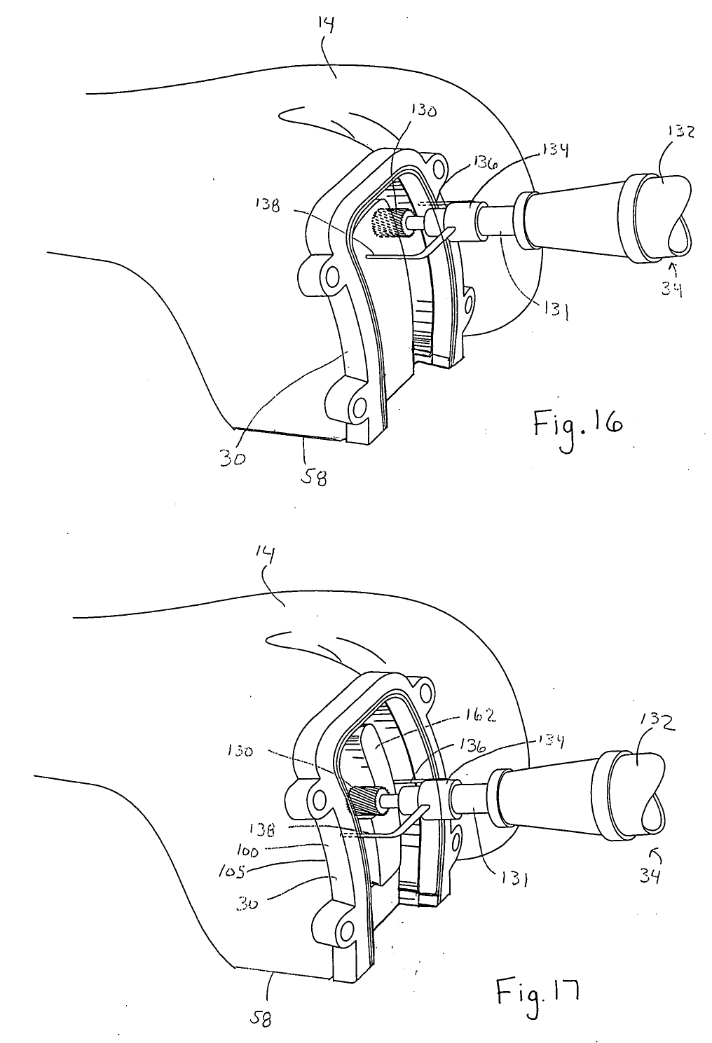

As shown in FIGS. 12-13 and 16-19, the femoral jig 30 may be a tracing block that is pinned to the femur 14 in a plurality of locations. The jig 30 matches the sizes of the femoral implants, and generally includes an upper portion 100, a lower portion 102, a front surface 104 and a rear surface 105. The tibial block is used to align the femoral jig properly with parallel surfaces aligned on the opposing tibial and femoral instruments. The upper portion 100 is generally upside down “U-shaped”, and includes a number of protrusions 108 about its periphery. The protrusions 108 each include a hole 110 for receiving a pin 112 for pinning the jig 30 to the femur 14. The upper portion 100 includes a cutout 114 extending through the jig 30 and generally defining the inside of the U-shape, and further includes a groove 116 that extends around the perimeter of the cutout 114 for engaging a milling tool. The rear surface 105 of the femoral jig, which will be in contact with the surface of the host femur 14, is contoured to accommodate for the rounded chamfer 48 of the lower portion of the curved femoral prosthesis. As shown in FIG. 14, the lower portion 102 includes a cutting slot 106 extending through the jig 30. The cutting slot 106 is capable of receiving a conventional saw blade 118, or a punch (not shown). When the femoral jig 30 is pinned to the femur 14, the cutting slot 106 is aligned with what will be the lower surface 58 of the femur 14, such that the saw blade 118 can be inserted through the slot 106 to resect the lower portion 120 of the host femur. In the illustrated embodiment, the lower portion 102 of the femoral jig 30 is removable from the upper portion 100, for instance, by magnetism, such that the lower portion 102 and resected bone 120 can be removed from the surgical area after the saw cut.

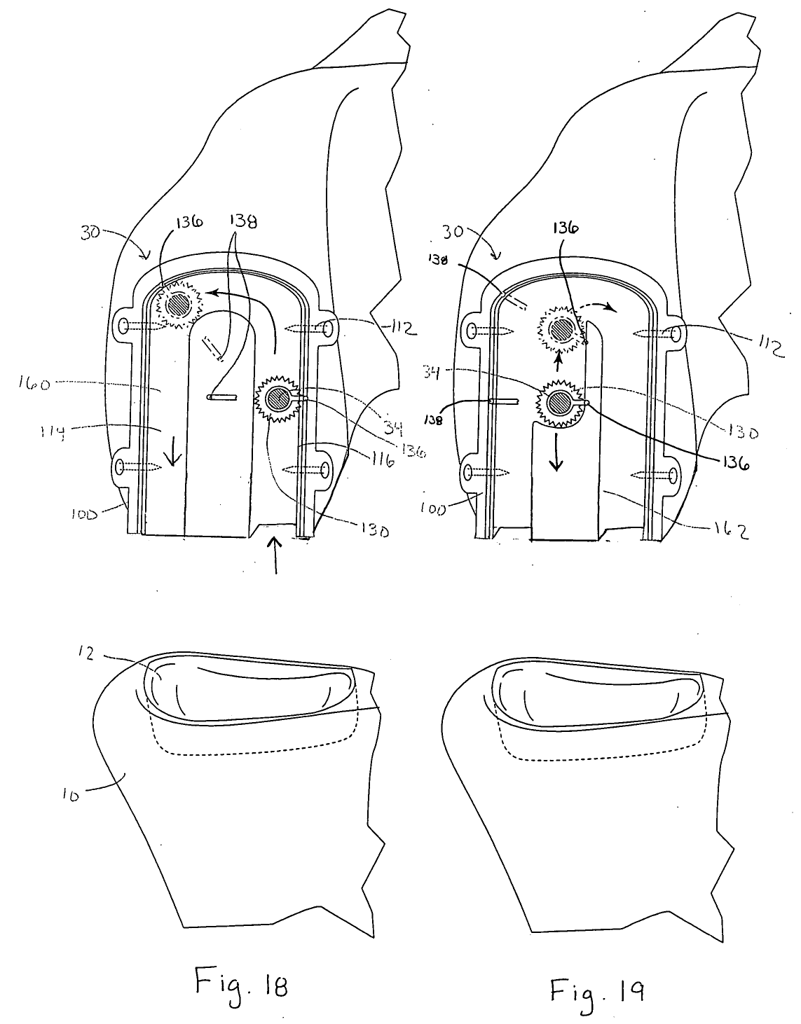

As with the tibial block 20, a power milling tool 34 is adapted to properly engage the femoral jig 30 for preparation of the host femur 14. As shown in FIGS. 13, 16 and 17, this milling tool 34 includes a standard burr 130 that extends from the neck 131 in line with the power handle 132, such that in operation the burr 130 can be actuated to rotate about the longitudinal axis of the handle 132. In one embodiment, a sleeve 134 is designed to frictionally or otherwise snap-fit around the neck 131 of the milling tool 34. Alternatively, the sleeve may be attached by screws or other fasteners. The sleeve 134 may be adjustable by sliding along the neck 131, and includes a pair of arms 136, 138 extending parallel to the burr spaced at different distances from the longitudinal axis of the handle 132. In the illustrated embodiment, the first arm 136 is closer to the longitudinal axis of the handle 132 than the second arm 138. The arms 136, 138 are sized to fit into the perimeter track or groove 116 on the femoral jig 30, such that as the arms are directed along the U-shaped track 116, the burr 130 extends through the cutout 114 and mills out the bone bed 60 in the host femur 14. As shown in FIGS. 18 and 19, the arms 136, 138 are spaced such that the burr 130 mills a first section of perimeter bone bed 60 when the first arm 136 is engaged with the groove 116 and a second section of bone (the remaining central bone island) when the second arm 138 is engaged with the groove 116.

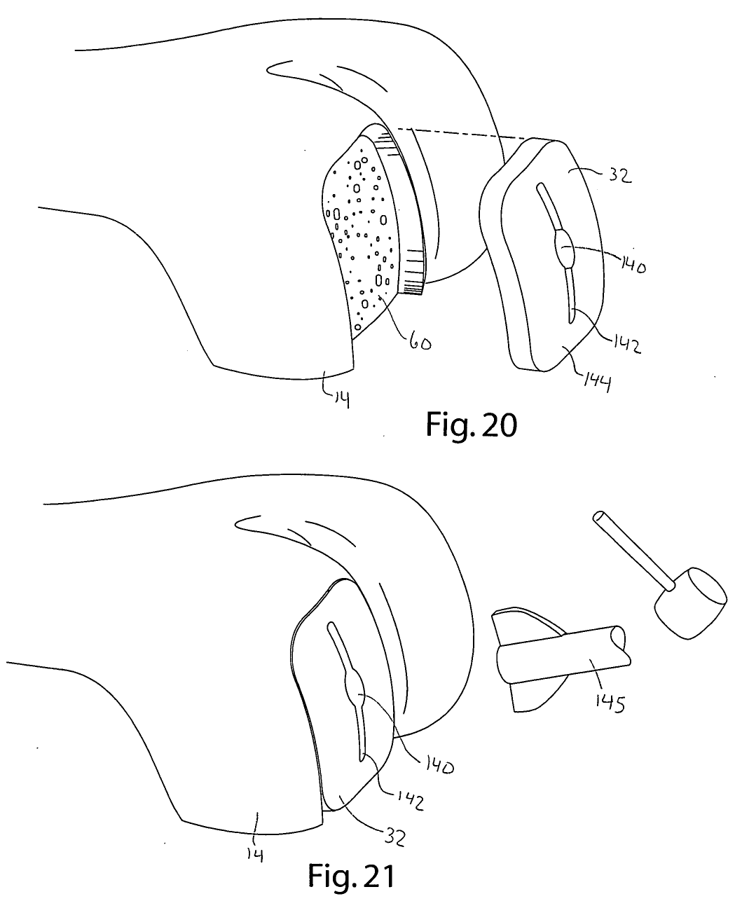

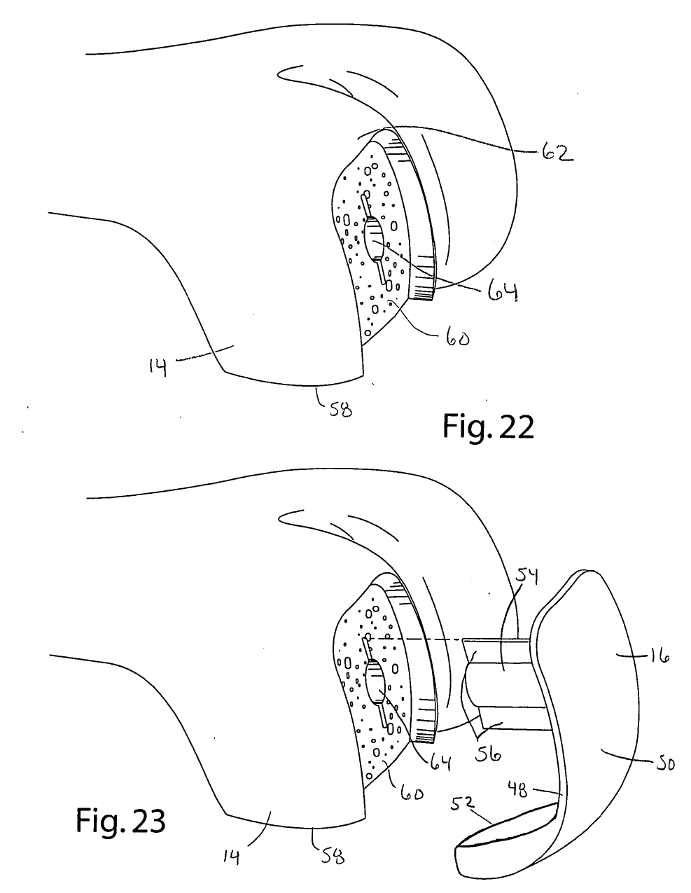

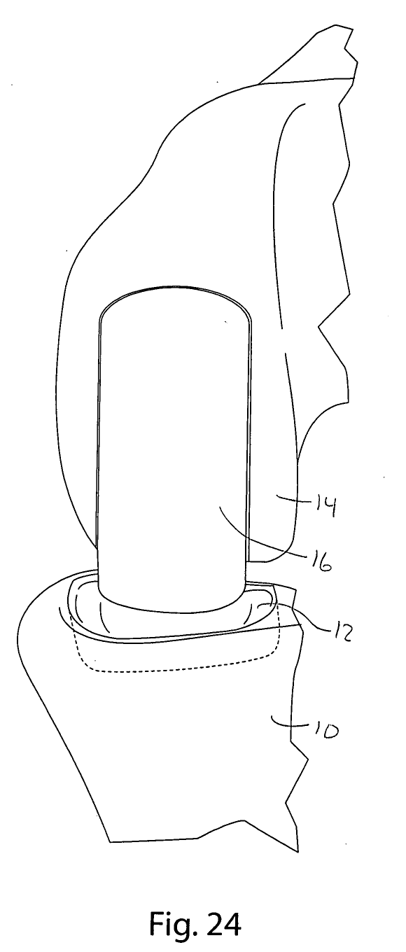

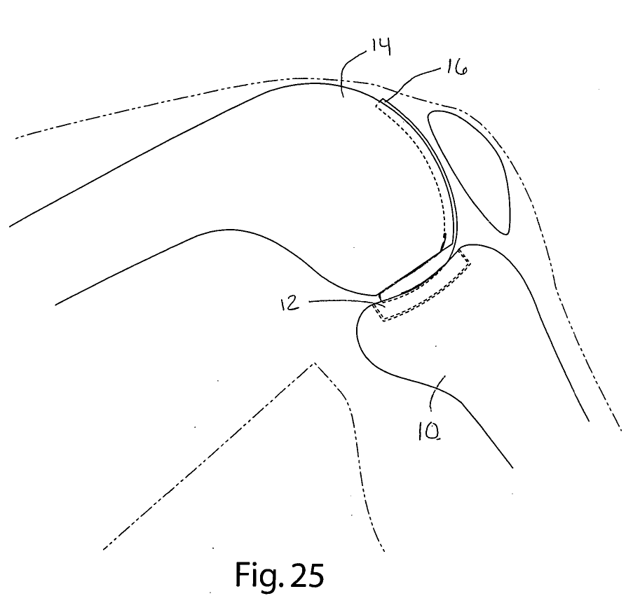

The femoral jig insert 32, as shown in FIGS. 20 and 21, is a plate that is shaped to fit inside the cutout 114 of the femoral jig 30 and into the milled out bone bed 60 of the femur 14. FIGS. 20 and 21 show the insert 32 fitted in the bone bed 60 after the femoral jig 30 has been removed from the femur 14, however, the insert 32 may alternatively be fitted into the bone bed 60 when the femoral jig 30 is pinned against the host femur 14. In one embodiment (not shown), the insert 32 may include a rim for engaging the perimeter groove 116 of the femoral jig 30 when the insert 32 is used with the femoral jig 30 still pinned to the femur 14. The insert 32 includes a central drilling hole 140, and a pair of slots 142 that extend from opposing sides of the drilling hole 140. The drilling hole 140 may further include a tubular protrusion (not shown) extending from the outer surface 144 of the insert 32 to act as a drill guide and stop. The slots 142 are designed to receive a fin punch 145, such as that shown in FIG. 21, or a standard saw blade for drilling and cutting the slot 64 to receive the protrusion 54 and fins 56 on the femoral prosthesis 16.

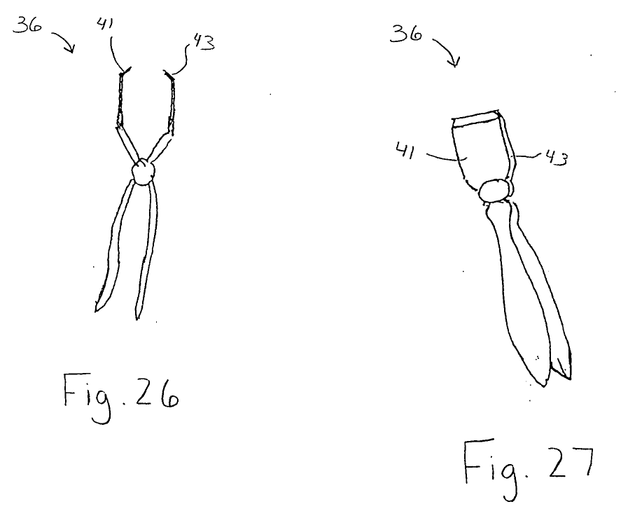

The femoral trial removal clamp 36, as shown in FIGS. 26 and 27, is a two pronged lever for removing a femoral trial prosthesis, before cementing the final femoral implant 16. The femoral trial prosthesis has the same dimensions as the final femoral prosthesis 16, but is typically made of a different material. The clamp 36 includes sharp, angled fingers 41, 43 that can be forced together under the margins of opposing sides of the femoral trial to wedge the femoral trial out of the host bone.

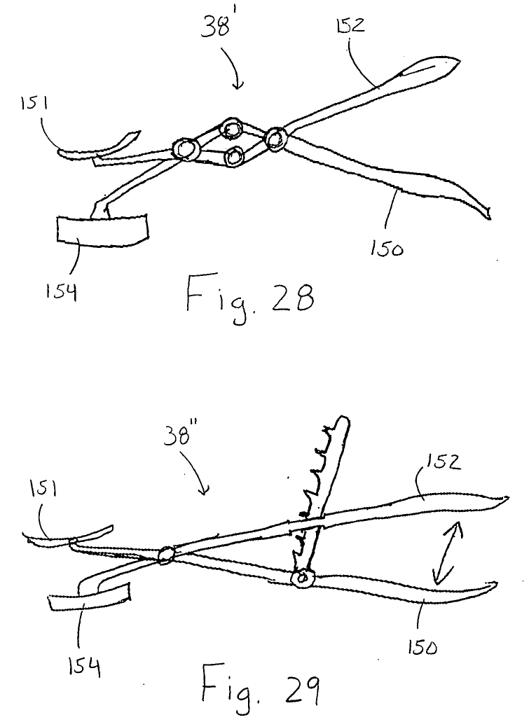

The spreader compression clamp 38, shown in FIG. 11, is a two pronged lever with first 150 and second 152 prongs that spread apart as the lever is compressed. The first prong 150 includes a pad 154 that is shaped similar to the tibial implant concave surface 42 to evenly contact the tibial implant 12. The second prong 152 includes a cupped end 151 to correspond with the shape of the outer surface the femoral prosthesis 16. The clamp serves to maintain compression at the finial implant-bone interface as bone cement hardens. FIGS. 28 and 29 show alternative embodiments 38′, 38″ of the spreader compression clamp 38. The embodiment shown in FIG. 28 includes a conventional double action hinge to decrease the force necessary to compress the arms 150 and 152. The embodiment shown in FIG. 29 includes a ratchet that maintains compression of the clamp 38″ without requiring constant force by the surgeon.

The present invention also includes a method for performing a unicompartmental knee replacement surgery. Again, the described embodiment is designed specifically for use with the Repicci UKR, and will be described as such. The method may be readily adapted for use with other knee replacements. The method comprises the steps of: a) pinning a tibial block 20 including a guide 24 or guides to the host tibia 10; b) adapting a milling tool 22 to engage the guide 24 on the tibial block 20; c) pinning a femoral milling jig 30 to the host femur 14, the jig 30 including a cutting slot 106 and a perimeter track 116; d) preparing the host femur 14 to receive the femoral implant 16 using the femoral milling jig 30 to guide cutting and milling tools that are adapted to attach to the jig 30; e) milling the surface of the host tibia 10 by directing the adapted milling tool 22 along the tibial block guide 24; f) inserting trial femoral and tibial prostheses into the respective bone beds and removing the femoral prosthesis using the femoral trial removal clamp, and g) compressing the final tibial 12 and femoral 16 implants with a spreader compression clamp 38.

The pinning of the tibial block preferably includes that the block is attached to a standard tibial extramedullary alignment jig 66, such as the Vanguard jig made by Biomet, Inc., shown in FIG. 1. The cuff 74 and lower portion 70 of the extramedullary jig 66 is externally secured to the ankle, and the tibial block 20 is secured on the tibia bone 10 by pins 78 that extend through a front portion 80 of the tibial block 20. When the tibial block 20 is pinned to the tibia 10, the contoured front portion 80 rests against the tibia 10.

The milling tool 22 may be adapted to engage the guide 24 by adding a pair of guide pegs 92 to the powered handle 86, such as by inserting the guide pegs 92 into holes on the handle 86, or by attaching a separate assembly that includes the guide pegs 92. As illustrated, the typical straight milling tool is converted to have the burr 90 angled at 90 degrees from the powered handle 86.

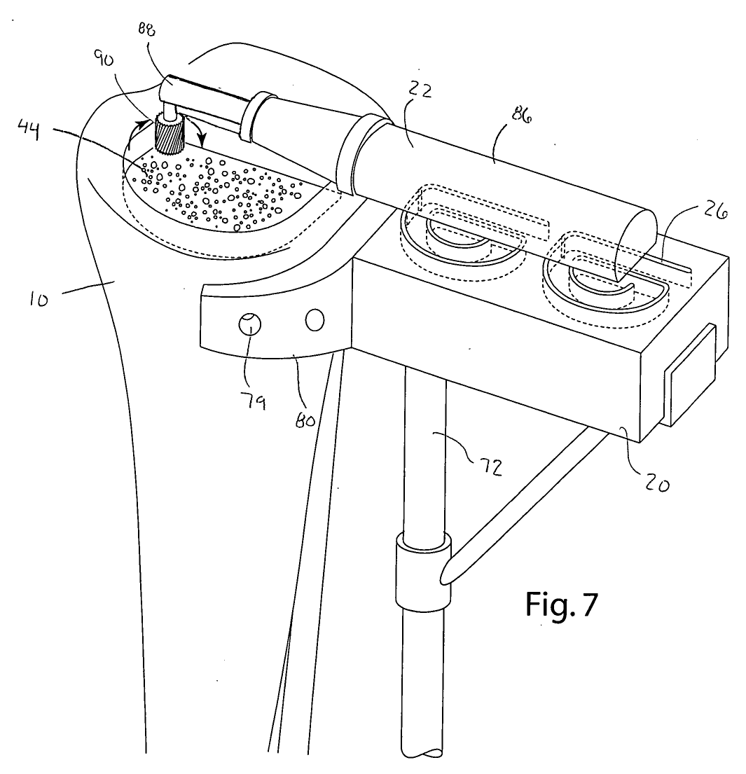

As shown in FIGS. 7 and 8, the milling of the host tibia 10 further includes engaging the guide pegs 92 on the milling tool 22 with the guides 24 on the tibial block 20, and then directing the guide pegs 92 within the guides 24 such that the burr 90 mills the desired pattern into the host tibia 10. As shown in FIG. 7, in the embodiment where the guides are a pair of grooves 26 in the tibial block 20, the pegs 92 may be engaged with the grooves 26 such that one peg fits in each groove. A surgeon may then trace the pegs 92 within the grooves 26 so the burr 90 mills out the bone bed 44 in the upper surface 46 of the tibia 10. Alternatively, as shown in FIG. 8, in the embodiment where the guide 24 is a pair of troughs 28 in the tibial block 20, the pegs 92 may each be inserted into one of the troughs, and the surgeon may move the pegs 92 about the troughs 28 until the burr 90 has milled out the bone bed 44. In either embodiment, the guide pegs 92 may be adjusted (or interchanged for an attachment of a different size) prior to milling to accommodate for the desired depth, size, and location of the bone bed 44.

Preparation of the host femur 14 may include 1) cutting a lower portion 120 of the host femur 14 by extending a saw blade or a punch 118 through a cutting slot 106 on the femoral jig 30, 2) removing the cut portion 120 of femur 14 and removable lower portion 102 of the femoral jig 30; and 3) engaging the milling tool 34 with the perimeter U-shaped track 116 on the femoral jig 30.

The milling tool 34 is adapted to engage the track 116 by attaching a sleeve 134 over the neck 131. The milling tool 34 includes a pair of arms 136, 138 extending parallel to the powered handle 132 and burr 130 and spaced at different distances from the burr 130. Referring now to FIGS. 16 and 18, a first arm 136 is placed into the track 116 and traced around the cutout 114 to mill out the bone in the perimeter portion 160 of the cutout 114. FIG. 18 shows the path of the burr 130 as the first arm 136 traces around the cutout 114. The first arm 136 is then removed from the track 116 and the second arm 138 is placed into the track 116 and traced about the track 116 so that the bone in the central portion 162 of the cutout 114 is milled out. FIGS. 17 and 19 show the second arm 138 engaged with the track 116. FIG. 19 shows the path of the burr 130 as the second arm 138 is traced around the track 116.

After the host bones have been milled, the surgeon may test that the tibial bone bed 44 and the femoral bone bed 60 have been properly sized by placing a trial tibial prosthesis (not shown) and a trial femoral prosthesis (not shown) in place in the respective beds. The trial prostheses are conventional and have the same size and shape as the final prostheses. With the trial prostheses in place, the surgeon may test the knee for a proper range of motion and stability. The femoral trial removal clamp 36 is then used to remove the embedded femoral trial prosthesis. The angled arms 41, 43 wedge underneath the edges of the trial on opposite sides of the trial to carefully remove the trial without damaging bone. The tibial trial component may be removed manually.

After the trial prostheses are removed, the final prostheses are typically set in place in the beds 44 and 60 with conventional bone cement. The spreader compression clamp is used to compress the final prostheses in place such that the tibial pad 154 presses against the tibial prosthesis and the cupped end 151 of the femoral prong 152 presses against the femoral prosthesis 16. The clamp is held in place while the cement cures.

The above description is that of a preferred embodiment of the invention. Various alterations and changes can be made without departing from the spirit and broader aspects of the invention as defined in the appended claims, which are to be interpreted in accordance with the principles of patent law including the doctrine of equivalents. Any reference to claim elements in the singular, for example, using the articles “a,” “an,” “the” or “said,” is not to be construed as limiting the element to the singular.

1. Instrumentation for aiding a surgeon in preparing a tibial bone bed in a host tibia and a femoral bone bed in a host femur for reception of a tibial prosthesis and a femoral prosthesis in a unicompartmental knee replacement surgery, comprising:
a tibial block fixed in position relative to the tibia, said tibial block including a tibial guide; and
a tibial rotational cutting device including a first portion and a second portion, said first portion adapted to engage said tibial guide, said second portion adapted to cut a desired portion of the host tibia when said first portion engages said tibial guide.

2. The instrumentation of claim 1 further comprising:
a femoral jig fixed in position relative to the host femur, said femoral jig including a femoral jig guide that is shaped to correspond with the shape of the femoral prosthesis; and
a femoral rotational cutting device including a first portion and a second portion, said first portion adapted to engage said femoral jig guide, said second portion adapted to cut a desired portion of the host femur when said first portion engages said femoral jig guide.

3. The instrumentation of claim 2 wherein said femoral jig includes an upper portion and a lower portion, said upper portion including said femoral jig guide, said lower portion including a slot for receiving a saw blade or a punch device.

4. The instrumentation of claim 2 wherein said instrumentation further includes a spreader compression clamp, said clamp including a lever having a first arm and a second arm, said first arm including a pad that is shaped to correspond with the upper surface of the tibial prosthesis, said second arm including an cupped end that is shaped to correspond with the lower surface of the femoral prosthesis, said cupped end and said pad spreading apart when an opposite end of each arm is compressed.

5. The instrumentation of claim 2 further comprising a femoral jig insert, said insert shaped to fit within said cut portion of said femur, said insert including a slot for receiving a cutting device.

6. The instrumentation of claim 5 wherein said femoral jig includes a cutout, said insert fitting within said cutout, said femoral jig guide including a track extending around said cutout.

7. The instrumentation of claim 6 wherein said femoral cutting device includes a handle, and a burr in-line with said handle, said cutting device including a plurality of arms, each of said arms spaced at a different distance from said burr, each of said arms fitting into said track and capable of being guided around said track, said burr cutting at least some of said desired cut portion of said femur when one of said arms is guided around said track.

8. The instrumentation of claim 1 wherein said first portion of said tibial cutting device includes a handle, and a plurality of pegs extending approximately perpendicular from said handle, said second portion of said device including a burr extending approximately perpendicular to said handle.

9. The instrumentation of claim 8 wherein said tibial guide includes a pair of grooves defined in said tibial block, said pegs fitting into said grooves and movable within said grooves, said grooves shaped such that said second portion of said tibial cutting device cuts said tibial bone bed when said pegs are moved within said grooves.

10. The instrumentation of claim 9 wherein said grooves define a spiral pattern.

11. The instrumentation of claim 8 wherein said tibial guide includes a pair of troughs, said troughs having the same shape as the tibial bone bed to be cut, said pegs each fitting into one of said troughs and movable within said trough, said second portion of said cutting device cutting said tibial bone bed when said pegs are moved in said troughs.

12. Instrumentation for aiding surgeons in the preparation of a tibial bone bed in a host tibia and a femoral bone bed in a host femur for the insertion of a tibial prosthesis and a femoral prosthesis in a unicompartmental knee replacement comprising:
a tibial block secured to the tibia, said tibial block including a guide that is shaped to correspond with the shape of the tibial prosthesis;
a tibial milling tool having a rotating burr and a handle, said burr extending from said handle and rotating about an axis perpendicular to said handle, said tibial milling tool including a plurality of pegs extending from said handle, said pegs engaging said guide and movable within said guide, said burr milling out said bone bed as said pegs are moved in said guide;
a femoral jig secured to the host femur, said femoral jig including a track that is shaped to correspond with the perimeter of the femoral prosthesis; and
a rotational milling tool including a burr, a handle, and a plurality of arms extending from said handle, each said arm fitting into said track and movable around said track, said burr milling out a portion of the femoral bone bed when each said arm is moved around said track.

13. The instrumentation of claim 12 wherein said guide in said tibial block includes first and second grooves, each said groove defining a spiral shaped path, each of said pegs fitting into one of said grooves, said burr milling out the bone bed when said pegs are moved within said grooves along said path.

14. The instrumentation of claim 12 wherein said guide in said tibial block is a pair of troughs, said troughs having a shape corresponding to the shape of said tibial bone bed, each of said pegs fitting into one of said troughs and movable throughout said trough, said burr milling out said bone bed when said pegs are moved throughout said troughs.

15. The instrumentation of claim 12 wherein said femoral jig includes a cutout, said track extending around said cutout, said burr extending through said cutout when said arms are fitted into said track.

16. The instrumentation of claim 15 wherein said femoral jig includes a femoral jig insert, said insert fitting through said cutout and including a slot, said slot receiving a saw blade or a punch.

17. The instrumentation of claim 12 wherein said tibial block is attached to a tibial extramedullary alignment jig.

18. The instrumentation of claim 12 wherein said femoral milling tool includes a first arm and a second arm, said arms spaced at different distances from said burr, such that said bur mills out a first portion of said femoral bed when said first arm is moved about said track and a remaining portion of said femoral bed when said second arm is moved about said track.

19. The instrumentation of claim 12 further including a spreader compression clamp, said clamp including a lever having a first arm and a second arm, said first arm including a pad that is shaped to correspond with the upper surface of the tibial prosthesis, said second arm including an cupped end that is shaped to correspond with the lower surface of the femoral prosthesis, said cupped end and said pad spreading apart to press said tibial and femoral prostheses into said bone beds when an opposite end of each arm is compressed.

20. The instrumentation of claim 12 wherein said femoral jig includes an upper portion and a lower portion, said upper portion attached to said femur, said lower portion removable from said upper portion and including a slot to receive a punch or a saw blade for resecting a lower portion of said femur.

21. The instrumentation of claim 12, further comprising a femoral trial removal clamp, said femoral trial removal clamp including first and second arms with angled ends.

22. A method for performing a unicompartmental knee replacement, including the steps of:
a) securing a tibial block including a guide or guides to a host tibia;
b) adapting a milling tool to engage the guide on the tibial block;
c) securing a femoral jig to a host femur;
d) adapting a milling tool to engage the femoral jig;
e) preparing the host femur to receive a femoral implant using the femoral milling jig to guide the milling tool adapted to engage the femoral jig;
f) milling a bone bed in the surface of the host tibia by directing the milling tool adapted to engage the tibial block along the tibial block guide; and
g) inserting a femoral prosthesis into the femoral bone bed and inserting a tibial prosthesis into the tibial bone bed.

23. The method of claim 22 further comprising compressing the tibial and femoral implants with a spreader compression clamp.

24. The method of claim 21 further comprising inserting a femoral trial prosthesis into the femoral bone bed and removing it with a femoral trial removal clamp.
